(19) United States
(12) Patent Application Publication
(10) Pub. No.: US 2025/0279162 A1
(43) Pub. Date: Sep. 4, 2025

(54) METHOD AND APPLICATION FOR CONSTRUCTING GENOMIC SELECTION MODEL BASED ON MULTI-TRAIT PHENOTYPING MODELING

(71) Applicants: Chinese Academy of Agricultural Sciences, Crop Science Institute, Beijing (CN); The Shennong Laboratory, Zhengzhou (CN)

(72) Inventors: Lijuan Qiu, Beijing (CN); Jun Wang, Zhengzhou (CN); Yijie Chen, Zhengzhou (CN); Shirui Teng, Zhengzhou (CN)

(21) Appl. No.: 19/031,201

(22) Filed: Jan. 17, 2025

(30) Foreign Application Priority Data
Mar. 4, 2024 (CN) 202410240643.7

(51) Int. Cl. G16B 20/40; G16B 10/00; G16B 40/00

(52) U.S. Cl. CPC G16B 20/40; G16B 10/00; G16B 40/00

(57) ABSTRACT

A method and system for constructing a genomic selection model based on a multi-trait phenotypic model are provided, the multi-trait model founded on phenotypic data, a predicted value or an estimated breeding value obtained by the genomic selection model is used as input data of a machine learning phenotypic prediction model to predict a final phenotypic value for line selection; a multi-trait machine learning phenotypic model is established to capture a linear or non-linear relationship between plant phenotypic traits, and on this basis, a predicted value and an estimated breeding value of each trait are obtained by combining it with the genomic selection model. The method enhances the prediction accuracy of target traits and accelerates the breeding process, improves the selection efficiency of the target traits and saves breeding costs, which is widely employed in the field of agricultural animal and plant breeding.

### CROSS-REFERENCE TO RELATED APPLICATIONS

This application claims priority to Chinese Patent Application No. 202410240643.7, filed Mar. 4, 2024, which is herein incorporated by reference in its entirety.

### TECHNICAL FIELD

The disclosure relates to the field of animal and plant breeding technologies, and more particularly to a method for constructing a genomic selection model based on multi-trait phenotyping modeling and its application.

### BACKGROUND

Genomic selection (GS) is a method that utilizes genome-wide markers to perform individual genetic evaluations to obtain genomic estimated breeding values (GEBV). It is mainly based on linkage disequilibrium (LD), assuming that each quantitative trait loci (QTL) that affects quantitative traits is in linkage disequilibrium with at least one marker and can explain most of the genetic variance. By constructing a prediction model, GS can calculate genomic estimated breeding values, thereby making predictions and selections in early individuals, effectively shortening the generation interval, improving selection accuracy and breeding efficiency, promoting the breeding process, and saving costs. In addition, GS shows good prediction effects when dealing with complex traits with low heritability and difficult to measure, truly realizing the use of genomic technology to guide breeding practices. Although GS was first proposed in the field of animal breeding, it has now made important progress in the field of plant breeding and has been widely used in crops such as Arabidopsis thaliana, maize, wheat, barley, and soybeans.

As the core of GS, statistical models greatly affect the accuracy and efficiency of genomic prediction. According to various selection models, GS is mainly divided into the following categories: a direct method, an indirect method and GS based on machine learning or deep learning. The direct method takes individuals as random effects, and the kinship matrix constructed by the genetic information of the training population and the validation population as the variance-covariance matrix. The variance components are estimated by the iterative method, and then the mixed linear model is solved to finally obtain the estimated breeding value of the individual to be predicted. The direct method is represented by genomic best linear unbiased prediction (GBLUP), which has high computational efficiency but slightly poor computational accuracy. The indirect method estimates the marker effect in the training population, and then accumulates the marker effect in combination with the genotype information of the prediction population, and finally obtains the estimated breeding value of the individual in the population. It is represented by ridge regression best linear unbiased prediction (rrBLUP), Bayesian model A (BayesA), Bayesian model B (BayesB), Bayesian model C (BayesC) and Bayesian least absolute shrinkage and selection operator (LASSO), which have huge computational complexity and slow speed. The GS model based on machine learning and deep learning can learn the relationship between genotype and phenotype in the training population from existing samples, and infer the phenotypic value based on the genotype data of the validation population. It is represented by crop genomic breeding machine (CropGBM), DeepGS, and deep neural network for genomic prediction (DNNGP). These methods consider multiple interactions and correlations between features, have high computational efficiency and high accuracy.

With the continuous improvement and optimization of GS statistical models, the stability of the models has been continuously improved and the types have been continuously enriched. However, there are still some problems and defects in the prior art. At present, most GS models predict and select for a single trait, ignoring the genetic basis between multiple related traits. The combined phenotypes of multiple traits can not only obtain the genetic correlation between traits, but also obtain the environmental correlation between traits, which is expected to improve the accuracy of phenotypic prediction. Although the selection index (SI)-based auxiliary prediction of target traits can use the genetic correlation between traits to construct a comprehensive index for the joint selection of multiple traits, this method has limited prediction effect on target traits with high heritability, and the use of auxiliary traits that are irrelevant to the target traits has the risk of reducing the predictive power of genomic selection for the target traits, and the selection of auxiliary traits is strict. In addition, as the number of auxiliary traits increases, the requirements for computing power will also increase. Therefore, it is urgent to build a genomic selection model with higher prediction accuracy, lower computing power requirements, and more stable prediction effects.

Through the above analysis, the problems and defects of the prior art are as follows:

(1) Currently, most GS models focus on predicting and selecting a single trait, ignoring the genetic and environmental correlations between multiple traits. This method of predicting the single trait cannot fully utilize the genetic correlations between multiple traits, thus limiting the accuracy of prediction and the effect of selection.

(2) The selection index can be used to combine the genetic correlations of multiple traits for joint selection. This method is limited in its effectiveness when predicting target traits with high heritability, and has strict requirements for the selection of auxiliary traits. The more auxiliary traits there are, the greater the computational burden.

### SUMMARY

In response to the gaps in the prior art, the disclosure provides a method for constructing a genomic selection model based on multi-trait phenotyping modeling and its application.

The disclosure establishes a machine learning model (also referred to as multi-trait model, or machine learning phenotypic prediction model, or multi-trait machine learning phenotyping model, or multi-trait phenotyping model) based on multi-trait phenotypic data (also referred to as the phenotypic data) to capture linear or nonlinear relationships between traits, and then inputs predicted values or estimated breeding values of multiple traits obtained by a genomic selection prediction model (also referred to as the genomic selection model) into the multi-trait phenotyping model to predict the target trait phenotype.

The target trait phenotype may be represented by the predicted value and the estimated breeding value of the target trait, after obtaining the predicted value and the estimated breeding value of the target trait, the method further includes: selecting and cultivating quality breeds based on the predicted value and the estimated breeding value of the target trait, for example, best individuals are selected for culture and reproduction.

Furthermore, the method for constructing the genomic selection model based on the multi-trait phenotyping model includes the following steps:
step 1: establishing the multi-trait machine learning phenotyping model; and
step 2: combining the genomic selection model with the multi-trait machine learning phenotyping model.

Furthermore, the above step 1 first corresponds to the order of the phenotypic data of each trait input into the multi-trait machine learning phenotyping model and the order of the predicted values and estimated breeding values of each trait obtained by the genomic selection model to ensure the consistency of the data and the validity of the linear or nonlinear relationships between traits. The multi-trait machine learning phenotyping model can select linear regression, logistic regression, support vector machine, decision tree or random forest according to the target trait.

Furthermore, the establishing the multi-trait machine learning phenotyping model includes the following steps:
(1) identifying and cleaning missing and abnormal values in the phenotypic data;
(2) randomly rearranging an order of lines in the phenotypic data;
(3) separating a target trait in the phenotypic data and normalizing remaining traits as feature values of the target trait to obtain normalized trait feature values of each line; and
(4) using the normalized trait feature values of each line as input, and training the model according to actual model parameters and hyperparameters of the multi-trait machine learning phenotyping model; and according to performance measurement of predicted phenotypic value results and target trait values, further adjusting various parameters of the model, and then storing a phenotypic prediction model with the best performance measurement as the multi-trait machine learning phenotyping model.

Furthermore, the combining the multi-trait machine learning phenotyping model with the genomic selection model specifically includes the following steps (see FIG. 1A and FIG. 1B):
1) selecting the genomic selection model to predict all traits separately; the genomic selection model can be GBLUP, BayesA, BayesB, BayesC, Bayesian LASSO, DNNGP or CropGBM;
2) performing data cleaning, processing and normalization on the predicted values or estimated breeding values of the traits;
3) the predicted values or estimated breeding values after the above data cleaning, processing and normalization are expressed as the feature values of the target trait;
4) loading the phenotypic prediction model and inputting the feature values for prediction; and the phenotypic prediction model makes accurate prediction based on the input feature values and outputs the final phenotypic prediction value that is close to the target trait.

The disclosure aims to provide a system according to the method for constructing a genomic selection model based on multi-trait phenotype modeling, which includes the following units:
a data preprocessing unit, configured to receive and process the phenotypic data, including identifying and cleaning missing and abnormal values in the phenotypic data, and randomly rearranging an order of lines;
a data normalization unit, configured to normalize non-target traits in the phenotypic data as feature values and separate a target trait;
a machine learning training unit, configured to train and store the multi-trait machine learning phenotyping model according to the normalized trait feature values by setting appropriate model parameters and hyperparameters;
a genomic selection model unit, configured to use genomic markers to perform genetic evaluation of individuals to obtain genomic estimated breeding values; and
a model integration unit, configured to combine the multi-trait machine learning phenotyping model and the genomic selection model and perform data cleaning, processing, and normalization of predicted values and estimated breeding values.

In an embodiment, each of the data preprocessing unit, the data normalization unit, the machine learning training unit, the genomic selection model unit and the model integration unit is a software module embedded and/or saved in one or more storage mediums and executable by one or more processors.

Furthermore, the machine learning training unit is configured to adjust the model according to the performance measurement of the predicted phenotypic value results and the target trait value, and store the phenotypic prediction model with the best performance measurement performance.

Furthermore, the model integration unit is configured to use the phenotypic prediction model, input normalized feature values for accurate prediction, and output final phenotypic prediction values that is close to the target trait.

The purpose of the disclosure is to provide a method combining a machine learning algorithm with a genomic selection model to improve the prediction accuracy and selection efficiency of target traits.

Another purpose of the disclosure is to provide a method for the genomic selection using multi-trait phenotypic data, so as to realize more scientific and efficient breeding selection.

In combination with the above technical solutions and the technical problems solved, the advantages and positive effects of the technical solutions to be protected by the disclosure are as follows.

First, the disclosure makes comprehensive use of multi-trait data for joint analysis, which can effectively utilize the genetic correlation and environmental correlation between traits. The disclosure uses multi-trait phenotypic data to establish a machine learning phenotyping model to capture the linear or nonlinear relationship between traits, and then inputs the predicted values or estimated breeding values of the traits obtained by the genomic selection model into the machine learning phenotyping model to predict the final phenotypic value and perform line selection. This integrated model aims to simultaneously utilize the correlation between multiple traits and between genotypes and traits, combine machine learning and genomic selection, predict target traits, and guide line selection. The model has lower computing power requirements and more stable prediction effects, providing a more reliable scientific basis for breeding decision makers. In practical applications, combinations of different machine learning models and different genomic selection models can be selected. From the final prediction performance, it can be seen that this method is applicable to most traits at the same time.

Second, the disclosure uses multi-trait phenotypic data to provide a method for constructing a genomic selection model based on a multi-trait phenotyping model, which improves the prediction accuracy and selection efficiency of the target trait and is applicable to most traits. The disclosure can be widely used in the field of agricultural animal and plant breeding. In the field of plant breeding such as crops, trees, fruits and vegetables, it can be used to predict and select individuals or lines with excellent genetic characteristics. Similarly, in the field of animal breeding such as poultry, fish and animal husbandry, the target trait performance can be improved according to the relevant traits, the genetic gain can be increased, and the genetic improvement process can be accelerated.

Third, the technical solution of the disclosure fills the technical gap in the industry: at present, many genomic selection models mainly focus on predicting a single trait. Although there are a few methods that can utilize the genetic correlation between multiple traits, higher requirements are placed on the selection of auxiliary traits. The increase of auxiliary traits will significantly increase the demand for computing power. The disclosure comprehensively utilizes the correlation between multiple traits to construct a machine learning phenotyping model to capture the linear or nonlinear relationship between traits. It has low requirements on computing power and can select a model with excellent performance based on the actual computing power. There is no need to strictly select auxiliary traits. Compared with existing methods, the disclosure is not only more flexible, but also more efficient and practical in processing large-scale data, and has a wider range of applications.

The technical solution of the disclosure solves the technical problem that people have been eager to solve but have never succeeded in solving: the genomic selection model continues to develop, but still faces many challenges, one of which is prediction accuracy. The disclosure proposes the method for constructing the genomic selection model based on the multi-trait phenotyping model by combining a machine learning model with genomic selection. This method not only effectively improves the prediction accuracy and selection efficiency, but is also suitable for prediction or selection of most traits.

Fourth, the significant technical progress brought about by the method for constructing the genomic selection model based on multi-trait phenotyping modeling of the disclosure is reflected in the following aspects.

### 1. Integrating Multi-Source Data to Enhance Prediction Accuracy

By combining the predicted values and estimated breeding values of the respective traits obtained by the genomic selection model with the machine learning phenotypic prediction model, this method can make full use of the existing genetic information and phenotypic data. This integrated method helps to improve the prediction accuracy of the target trait, thereby improving breeding efficiency and success rate.

### 2. Capturing Complex Correlations Between Traits

The constructed multi-trait machine learning phenotyping model can capture linear and nonlinear relationships between traits, which is often difficult to achieve in traditional genomic selection models. By identifying these complex trait correlations, breeders can more accurately predict and select lines.

### 3. Refinement of Data Preprocessing and Model Optimization

Detailed data preprocessing steps (such as identifying and cleaning missing and abnormal values in the phenotypic data, random rearranging, etc.) and parameter adjustment of the machine learning model ensure the robustness and generalization ability of the model. Such detailed operations help improve the model's adaptability to new data and the accuracy of predicting phenotypic values.

### 4. Improving the Efficiency of Breeding Decisions

By using the machine learning model for phenotypic prediction, this method can quickly and accurately predict the final phenotypic value of the line, thereby providing scientific decision support for breeders. This efficient prediction capability can significantly shorten the breeding cycle and accelerate the cultivation and promotion of excellent varieties.

### 5. Flexibility and Scalability

The construction method is not only suitable for plant breeding, but can also be adjusted and applied to other biological breeding fields as needed. Its flexibility and scalability make the method have broad application prospects.

The method for constructing the genomic selection model based on the multi-trait phenotyping model of the disclosure has brought significant technological progress in improving prediction accuracy, capturing complex correlations between traits, improving breeding decision-making efficiency, and enhancing the flexibility and scalability of the method, providing an efficient and accurate selection tool for modern breeding.

### BRIEF DESCRIPTION OF DRAWINGS

To clearly explain technical solutions of embodiments of the disclosure, accompanying drawings in the embodiments of the disclosure are simply introduced below. Apparently, the accompanying drawings described below are only some embodiments of the disclosure, for those skilled in the art, other drawings can be obtained according to the accompanying drawings without creative labor.

FIGS. 1A-1B illustrate schematic diagrams of combining genomic selection with a machine learning multi-trait model provided by an embodiment of the disclosure.

FIG. 2 illustrates a schematic diagram of the prediction accuracy of soybean yield-related traits provided by an embodiment of the disclosure; GBLUP: genomic best linear unbiased prediction; SI: selection index; CropGBM: crop genomic breeding machine; MT-GBLUP: genomic selection model based on multi-trait phenotyping model and combining GBLUP, that is, multi-trait GBLUP; MT-CropGBM: genomic selection model based on multi-trait phenotyping model and combining CropGBM, that is, multi-trait CropGBM.

FIG. 3 illustrates a schematic diagram of the selection efficiency of yield-related traits of different methods provided in an embodiment of the disclosure; PS: phenotypic selection; CropGBM: genomic selection; MT-GBLUP: genomic selection model based on multi-trait phenotyping model and combining GBLUP; MT-CropGBM: genomic selection model based on multi-trait phenotyping model and combining CropGBM.

### DETAILED DESCRIPTION OF EMBODIMENTS

In order to make the purpose, technical solution and advantages of the disclosure more clearly understood, the disclosure is further described in detail below in conjunction with the embodiments. It should be understood that the specific embodiments described herein are only used to explain the disclosure and are not used to limit the disclosure.

In view of the problems existing in the prior art, the technical solution adopted by the disclosure is as follows.

Limitations of single-trait prediction: traditional genomic selection models mainly predict single traits, ignoring interrelationships between multiple traits. The disclosure establishes a multi-trait phenotyping model, which takes into account the interrelationships between multiple traits, including linear and nonlinear relationships, thereby providing a more comprehensive genetic evaluation.

Deficiencies in data processing and integration: the prior art has deficiencies in processing and integrating phenotypic data and the predicted values obtained by the genomic selection model. The data preprocessing steps (such as data cleaning and normalization) in the disclosure ensure the consistency and accuracy of the input data, providing a reliable basis for subsequent model training and prediction.

Low accuracy of model training and prediction: traditional GS models have limitations in accuracy and stability. The disclosure combines the machine learning technology to optimize the model training process, and adjusts parameters based on the performance measurement of the predicted phenotypic value results and the target trait values, thereby improving the accuracy and stability of the model.

The technical effects and significant technical progress brought about by the disclosure in solving the problems of the prior art are as follows.

Improved accuracy of joint prediction of multiple traits: by considering the interrelationships between multiple traits, the disclosure improves the prediction accuracy in the breeding process, making the selection of lines more precise and efficient.

Optimization of data processing: refined data preprocessing steps ensure the high quality of model input, thereby improving the reliability of the final prediction.

Improved flexibility and adaptability of the model: by combining different machine learning models and different genomic selection models, the disclosure can adapt to different phenotypic data and genetic backgrounds, providing a more flexible and adaptable breeding evaluation tool.

Significant improvement in breeding efficiency: using the method of the disclosure, line selection can be performed quickly and accurately at an early stage, thereby effectively shortening the breeding cycle and saving time and cost.

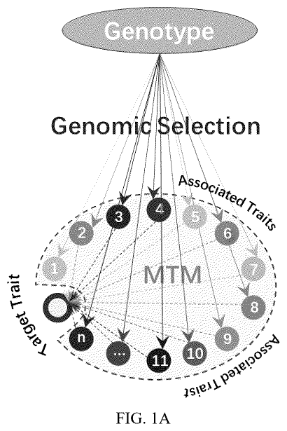

The disclosure provides a specific embodiment, which uses a soybean yield genomic selection model based on a multi-trait model of 75 traits. The embodiment individually combines a random forest model with GBLUP and CropGBM to construct two models, MT-GBLUP (Multi-Trait GBLUP) and MT-CropGBM (Multi-Trait CropGBM), to predict and select the seed weight per plant, the pod number per plant, the seed number per plant and the 100 seed weight, as shown in FIG. 1A and FIG. 1B. The model construction steps are as follows.

Step 1: a random forest phenotyping model is established based on multi-trait data.

(1) Missing values and abnormal values are identified and cleaned up in data from multiple lines, each containing 75 different traits, to improve data quality and the accuracy of predictions made by a machine learning phenotyping model.

(2) The order of soybean lines in the multi-line phenotypic data with 75 traits is randomly rearranged to eliminate or reduce potential sorting bias.

(3) The seed weight per plant, the pod number per plant, the seed weight per plant, and the 100 seed weight in the phenotypic data are respectively used as target traits of the random forest phenotyping model, and the remaining traits are normalized as the feature values of the target traits to improve the data processing efficiency in the machine learning phenotyping model and ensure that all features have similar scales, thereby accelerating the model training speed and improving the algorithm performance.

(4) The normalized trait feature values of the soybean lines are input into the phenotyping model, and the initial values of the model parameters and hyperparameters of the machine learning model are set according to the actual situation and the model is trained. According to the performance measurement of the predicted phenotypic value results and the target trait values, the parameters are further adjusted to optimize the model prediction result, and then the phenotypic prediction model with the best performance of performance measurement is stored.

Step 2: the random forest multi-trait phenotyping model is combined with GBLUP and CropGBM individually. This process not only enhances the prediction accuracy and improves the selection efficiency of key target traits, but also provides a more reliable scientific basis for breeding decisions.

(1) GBLUP and CropGBM are used to predict 75 traits of the soybean lines and obtain estimated breeding values and predicted values.

(2) As in step 1, data cleaning, processing and normalization are performed on the predicted values and the estimated breeding values, and the predicted values or the estimated breeding values after the above data processing operation are expressed as the feature values of the target trait.

(3) The phenotypic prediction model is loaded and the feature values are input for prediction. The model makes accurate prediction based on the input feature values and outputs the final phenotypic prediction values close to the target trait for soybean line selection.

### 1. A Genomic Selection Model for Soybean Yield Based on a Multi-Trait Phenotyping Model

Based on the random forest algorithm, this embodiment uses the phenotypic data other than yield-related traits to construct a multi-trait model, and uses the estimated breeding values or phenotypic values of GBLUP and CropGBM as the feature values of the multi-trait model. The results show that MT-GBLUP and MT-CropGBM improve the prediction accuracy and selection efficiency of the target traits. This shows that the combination of the multi-trait model and the genomic selection is expected to play an important role in breeding, help optimize crop yields, and improve agricultural production efficiency.

### 1.1 Phenotypic Analysis of 75 Agronomic Traits of Soybean

The test material is a soybean nested association mapping (NAM) population with ‘Zhongdou 41’ as the common female parent and the remaining 35 male parents coming from landrace and cultivar. The NAM population was planted in Jingzhou City, Hubei Province, China in the summer of 2020, with a total of 2,455 lines. According to the soybean germplasm resource data standard, 75 soybean agronomic traits were collected during planting and after harvest (see Table 1). The data related to the leaf shape, the seed shape and the seed color traits were collected using Wan Shen LA_S root analysis system and the SC-G automatic seed analysis system, as well as a dry seed weight meter, while the data on protein and oil content were obtained by the model scanning of Botong (DA7200) instrument.

The phenotypic data were subjected to the 3-sigma principle to remove the abnormal values, and then the Shapiro-Wilk method was used for normality test. Descriptive statistics and Pearson correlation analysis were performed using R packages such as Psych and Hmisc. The descriptive statistics showed that the variation range of the 75 traits was between 0.02 and 5925.24, and the coefficient of variation of 16 traits was greater than 30%, which was a high-variability trait (see Table 2). Specifically, the variation ranges of the seed weight per plant, the pod number per plant, the seed number per plant, and the 100 seed weight were 4.36-54.13, 14.40-147.80, 19.25-263.40, and 11.50-30.51, respectively, and the coefficients of variation were 32.78%, 36.97%, 34.52%, and 15.58%. The skewness and kurtosis and Shapiro-Wilk normality test results showed that the 75 traits were approximately subject to normal distribution (see Table 2). The results of correlation analysis showed that there was a significant positive correlation between the seed weight per plant, the seed number per plant and the pod number per plant (P<0.01), with correlations as high as 0.77-0.88, but the 100 seed weight was negatively correlated with both seed number per plant and pod number per plant, at −0.31 and −0.25, respectively, and the correlation with the seed weight per plant was 0.19. Similarly, the seed weight per plant, the seed number per plant and the pod number per plant were highly correlated with the pod number per plant, the main stem pod number, the branch length and the effective branch number, but the 100 seed weight was negatively correlated with these traits (see Table 3).

### 1.2 Prediction Accuracy of Soybean Yield-Related Traits

The genomic data are NAM population F8 chip data. The raw data are converted to variant call format (vcf), and the PLINK software is used to screen markers with integrity higher than or equal to 80% (—geno 0.20) and minor allele marker frequency lower than 0.05 (—maf 0.05). 103,966 SNP markers are left, and Beagle 5.4 software is used to impute in missing genotypes.

In order to measure the prediction ability of different methods, based on the phenotypic and genotypic data of yield-related traits, this study used GBLUP, SI, CropGBM, MT-GBLUP and MT-CropGBM to predict four yield-related agronomic traits, and used the Pearson correlation coefficient between the phenotypic value and the estimated breeding value/predicted value in the validation set as the prediction accuracy. GBLUP uses a five-fold cross-validation method, sets a random seed, iterates 100 times, and calculates the average prediction accuracy of multiple iterations. SI, based on GBLUP, uses four yield traits as target traits in turn, and uses traits other than the target traits as auxiliary traits to combine to construct the selection index and calculate the estimated breeding value of the target trait. CropGBM divides the data set into 80% training set and 20% validation set for prediction. MT-GBLUP and MT-CropGBM, based on the construction of random forest models using multi-trait phenotypic data, use estimated breeding values or phenotypic prediction values calculated by GBLUP and CropGBM to comprehensively predict four agronomic traits related to yield.

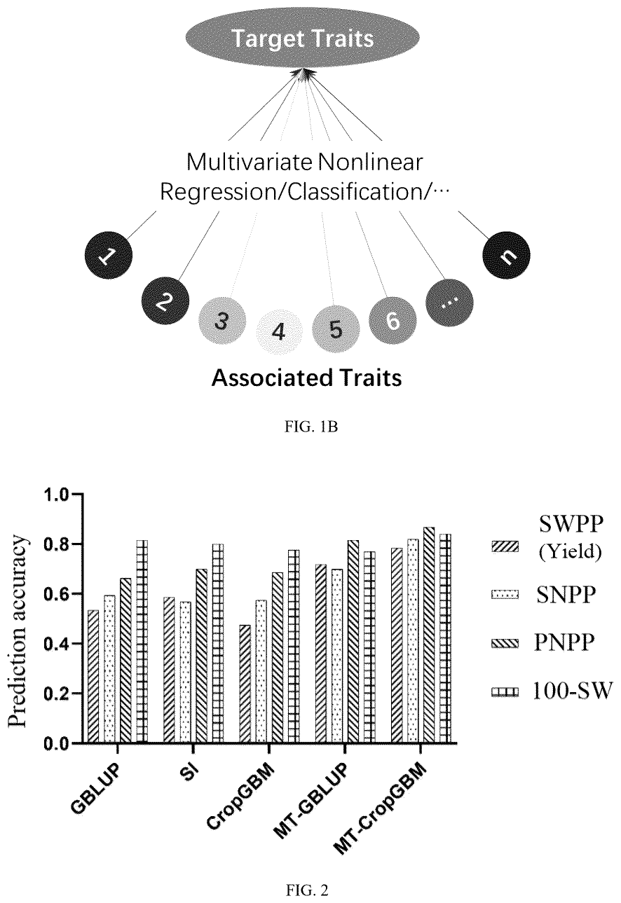

In the GBLUP prediction results, the prediction accuracy of seed weight per plant, seed number per plant, pod number per plant and 100 seed weight were 0.53, 0.59, 0.66 and 0.82, respectively (FIG. 2). Compared with GBLUP, the SI prediction accuracy of seed weight per plant and pod number per plant increased by 0.06 and 0.04, respectively, while the seed number per plant and 100 seed weight decreased by 0.02. Similarly, only the CropGBM prediction accuracy of the pod number per plant increased by 0.03, and the prediction accuracy of the other three traits decreased to varying degrees (0.02-0.06). In contrast to CropGBM, the MT-GBLUP prediction accuracy of seed weight per plant, seed number per plant and pod number per plant increased to 0.72, 0.70 and 0.82. In addition, MT-CropGBM has been further improved (FIG. 2). Compared with GBLUP, the improvement of the four traits is between 0.03 and 0.25. The reported yield GS prediction accuracy ranges from 0.26 to 0.72. In comparison, MT-CropGBM (0.78-0.87) shows a more significant improvement (see Table 4 and FIG. 2). From the comparison of the results of the above four prediction methods, it can be concluded that CropGBM alone has the worst prediction effect, but after combining with the multi-trait phenotyping model, the prediction ability is significantly improved. Although SI also utilizes the correlation between phenotypes, the improvement is lower and the accuracy of some traits decreases. MT-GBLUP and MT-CropGBM have excellent performance in most traits, with a large improvement. This shows that the method of combining the multi-trait model with GS proposed in the disclosure is expected to provide strong support for breeding work and has good prediction performance in yield-related traits.

### 1.3 Selection Efficiency of Soybean Yield

In order to evaluate the selection effect of different methods on high-yield soybean lines, high-yield soybeans are screened with the standards of 28 g seed weight per plant, 22 g 100 seed weight, 70 pod number per plant, and 130 seed number per plant, and the selection efficiency is calculated as follows:

$$\begin{matrix}Phenotypic selection : y = \frac{{x}_{r}}{{x}_{t}} \times 100 \% \\ CropGBM , MT ‐ GBLUP and MT ‐ CropGBM : y = \frac{{x}_{s}}{{x}_{m}} \times 100 \%\end{matrix}$$

Where y is the selection efficiency, xₜ is the number of all lines planted in Jingzhou City, Hubei Province, in China in 20 years, xᵣ is the number of high-yield lines obtained based on phenotypic selection analysis, xₘ is the number of high-yield lines obtained based on CropGBM, MT-GBLUP or MT-CropGBM prediction value analysis, and xₛ is the number of lines obtained using other prediction methods that are consistent with the high-yield lines obtained from phenotypic selection analysis.

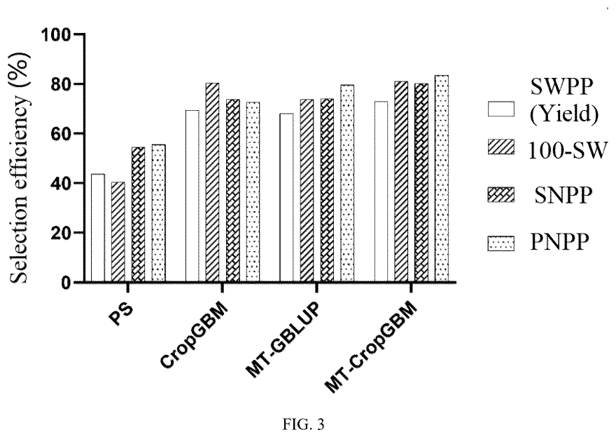

Based on phenotypic selection, 1072 seed weight per plant, 1115 seed numbers per plant, 1007 pod numbers per plant, and 993 100 seed weight lines exceeded the set high-yield thresholds, with selection efficiencies of 43.67%, 45.42%, 41.02%, and 40.45%, respectively. Similarly, based on the high-yield threshold, CropGBM predicted 1244, 1230, 1096, and 926 high-yield lines for seed weight per plant, seed number per plant, pod number per plant, and 100 seed weight, respectively, of which 864, 875, 783, and 744 are consistent with the high-yield lines selected by phenotypic selection, with selection efficiencies of 69.45%, 71.14%, 71.44%, and 80.35%, respectively (FIG. 3). Compared with CropGBM, the selection efficiency of the four yield traits in MT-GBLUP shows a downward trend, with a decrease ranging from 0.94% to 6.57%. In contrast to MT-CBLUP, the selection efficiency of seed weight per plant, seed number per plant, pod number per plant, and 100 seed weight in MT-CropGBM increase to 72.92%, 74.39%, 76.43% and 81.11%, respectively, with the best selection effect (FIG. 3). The method (MT-CropGBM) combining the multi-trait random forest model and CropGBM proposed in the disclosure can improve resource utilization efficiency and achieve higher production benefits by selecting high-yield lines.

TABLE 1
Categories, abbreviations and units of 75 agronomic traits

| | Full name of | | |
|---|---|---|---|
| Category | agronomic traits | Abbreviation | Unit |
| Yield | Seed weight per plant | SWPP | g |
| | Pod number per plant | PNPP | counts |
| | Seed number per | SNPP | counts |
| | plant | | |
| | 100 seed weight | 100-SW | g |
| Reproductive period | Flowering to | FTM | days |
| | maturation time | | |
| | Maturation time | MT | days |
| | Flowering time | FT | days |
| Stem | Internode number | IN | counts |
| | Stem diameter | SD | cm |
| | Plant height | PH | cm |
| | Podding habit | POH | |
| | Lodging | LG | |
| | Growth habit | GH | |
| Branch | Branch length | BL | cm |
| | Branch internode | BIN | counts |
| | number | | |
| | Branch diameter | BD | cm |
| | Effective branch | EBN | counts |
| | number | | |
| | Branch angle | BA | degree |
| Pod | Moldy pod number | MPN | counts |
| | Moldy pod number | MPNR | |
| | ratio | | |
| | Four seeded pod | FSPNPP | counts |
| | number per plant | | |
| | Branch pod number | BPN | counts |
| | Four seed ratio | FSR | |
| | Pod width | PW | mm |
| | Pod length | PL | mm |
| | First pod height | FPH | cm |
| | Stem pod number | SPN | counts |
| Seed | Seed perimeter | SP | mm |
| | Seed length | SL | mm |
| | Blue | B | |
| | Red | R | |
| | Green | G | |
| | Seed width | SW | mm |
| | Seed area | SA | mm² |
| | Seed length to width | SLWR | |
| | ratio | | |
| Top leaf | Top middle leaf | TMLL | mm |
| | length | | |
| | Top middle leaf | TMLP | mm |
| | perimeter | | |
| | Top side leaf | TSLP | mm |
| | perimeter | | |
| | Top side leaf length | TSLL | mm |
| | Top middle leaf area | TMLA | mm² |
| | Top side leaf width | TSLW | mm |
| | Top middle leaf | TMLW | mm |
| | width | | |
| | Top side leaf area | TSLA | mm² |
| | Top middle leaf | TMLLWR | |
| | length width ratio | | |
| | Top side leaf length | TSLLWR | |
| | width ratio | | |
| Middle leaf | Middle middle leaf | MMLP | mm |
| | perimeter | | |
| | Middle side leaf | MSLL | m |
| | length | | |
| | Middle side leaf | MSLP | m |
| | perimeter | | |
| | Middle middle leaf | MMLL | m |
| | length | | |
| | Middle middle leaf | MMLLWR | |
| | length width ratio | | |
| | Middle side leaf | MSLW | m |
| | width | | |
| | Middle side leaf area | MSLA | m² |
| | Middle middle leaf | MMLW | m |
| | width | | |
| | Middle side leaf | MSLLWR | |
| | length width ratio | | |
| Bottom leaf | Bottom side leaf | BSLP | m |
| | perimeter | | |
| | Bottom middle leaf | BMLL | m |
| | length | | |
| | Bottom middle leaf | BMLP | m |
| | perimeter | | |
| | Bottom side leaf | BSLL | m |
| | length | | |
| | Bottom middle leaf | BMLW | m |
| | width | | |
| | Bottom middle leaf | BMLA | m² |
| | area | | |
| | Bottom side leaf | BSLW | m |
| | width | | |
| | Bottom side leaf area | BSLA | m² |
| | Bottom side leaf | BSLLWR | |
| | length width ratio | | |
| | Bottom middle leaf | BMLLWR | |
| | length width ratio | | |
| Petiole length | Petiole length middle | PLM | m |
| | Petiole length bottom | PLB | m |
| | Petiole length top | PLT | m |
| | Petiole angle top | PAT | degree |
| | Petiole angle middle | PAM | degree |
| | Petiole angle bottom | PAB | degree |
| Quality | Protein content | PC | |
| | Oil content | OC | |
| | Protein and oil | POC | |
| | content | | |
| Flower | Flower color | FC | |

TABLE 2
Descriptive statistical analysis of 75 agronomic traits

| | Agronomic | Mean | Maximum | Minimum | | Standard | | | Coefficient of | W statistical | P |
|---|---|---|---|---|---|---|---|---|---|---|---|
| Category | trait | value | value | value | Range | deviation | Skewness | Kurtosis | variation (%) | value | value |
| Yield | SWPP | 27.82 | 54.13 | 4.36 | 49.77 | 19.12 | 0.43 | −0.19 | 32.78 | 0.98 | 0.00 |
| | PNPP | 69.35 | 147.80 | 14.40 | 133.40 | 25.64 | 0.76 | 0.20 | 36.97 | 0.96 | 0.00 |
| | SNPP | 133.20 | 263.40 | 19.25 | 244.15 | 45.98 | 0.59 | −0.12 | 34.52 | 0.97 | 0.00 |
| | 100-SW | 21.17 | 30.51 | 11.50 | 19.01 | 3.30 | −0.03 | −0.29 | 15.58 | 1.00 | 0.01 |
| Growth | FTM | 67.91 | 85.00 | 50.00 | 35.00 | 7.27 | 0.00 | −0.20 | 10.70 | 0.98 | 0.00 |
| period | MT | 108.20 | 131.00 | 86.00 | 45.00 | 9.43 | 0.18 | −0.10 | 8.71 | 0.94 | 0.00 |
| | FT | 33.52 | 39.00 | 29.00 | 10.00 | 2.85 | −0.20 | −0.71 | 8.50 | 0.86 | 0.00 |
| Stem | IN | 13.63 | 19.40 | 8.40 | 11.00 | 2.04 | 0.44 | −0.01 | 14.98 | 0.98 | 0.00 |
| | SD | 5.70 | 8.13 | 3.25 | 4.87 | 0.85 | 0.29 | −0.09 | 14.94 | 0.99 | 0.00 |
| | PH | 65.52 | 104.88 | 28.56 | 76.32 | 13.72 | 0.33 | −0.10 | 20.94 | 0.99 | 0.00 |
| | POH | 2.10 | 3.00 | 1.00 | 2.00 | 0.57 | −0.12 | −0.98 | 27.20 | 0.96 | 0.00 |
| | LG | 1.10 | 3.00 | 0.00 | 3.00 | 1.19 | 0.54 | −1.28 | 108.32 | 0.78 | 0.00 |
| | GH | 1.01 | 2.00 | 1.00 | 1.00 | 0.08 | 11.95 | 146.15 | 7.70 | 0.07 | 0.00 |
| Branch | BL | 21.49 | 43.13 | 4.43 | 38.70 | 7.42 | 0.35 | −0.21 | 34.54 | 0.99 | 0.00 |
| | BIN | 4.00 | 6.72 | 2.00 | 4.72 | 0.94 | 0.38 | −0.04 | 23.41 | 0.99 | 0.00 |
| | BD | 2.98 | 3.94 | 2.00 | 1.94 | 0.35 | 0.17 | −0.16 | 11.91 | 1.00 | 0.00 |
| | EBN | 4.28 | 8.40 | 0.40 | 8.00 | 1.37 | 0.27 | 0.17 | 31.97 | 0.99 | 0.00 |
| | BA | 17.58 | 27.13 | 8.12 | 19.01 | 3.38 | 0.23 | −0.23 | 19.25 | 0.99 | 0.00 |
| Pod | MPN | 10.97 | 34.00 | 0.00 | 34.00 | 7.88 | 0.92 | 0.13 | 71.79 | 0.92 | 0.00 |
| | MPNR | 0.15 | 0.48 | 0.00 | 0.48 | 0.11 | 0.88 | 0.12 | 69.80 | 0.93 | 0.00 |
| | FSPNPP | 0.23 | 1.40 | 0.00 | 1.40 | 0.36 | 1.69 | 2.01 | 153.15 | 0.70 | 0.00 |
| | BPN | 35.23 | 87.40 | 0.00 | 87.40 | 17.82 | 0.70 | 0.07 | 50.58 | 0.96 | 0.00 |
| | FSR | 0.00 | 0.02 | 0.00 | 0.02 | 10.01 | 1.79 | 2.41 | 158.37 | 0.69 | 0.00 |
| | PW | 10.11 | 11.80 | 8.39 | 3.41 | 0.62 | −0.02 | −0.20 | 6.10 | 1.00 | 0.02 |
| | PL | 4.59 | 5.57 | 3.60 | 1.98 | 0.36 | 0.10 | −0.21 | 7.78 | 1.00 | 0.00 |
| | FPH | 11.08 | 21.50 | 2.88 | 18.62 | 3.54 | 0.26 | −0.43 | 31.99 | 0.99 | 0.00 |
| | SPN | 33.52 | 59.80 | 11.80 | 48.00 | 9.42 | 0.48 | −0.29 | 28.11 | 0.98 | 0.00 |
| Seed | SP | 24.70 | 28.47 | 20.79 | 7.67 | 1.40 | 0.19 | −0.17 | 5.66 | 1.00 | 0.00 |
| | SL | 8.49 | 10.11 | 6.84 | 3.27 | 0.59 | 0.26 | −0.19 | 6.92 | 0.99 | 0.00 |
| | B | 43.92 | 124.00 | 10.60 | 113.40 | 37.11 | 0.67 | −1.43 | 84.48 | 0.71 | 0.00 |
| | R | 66.00 | 167.80 | 12.20 | 155.60 | 50.60 | 0.65 | −1.42 | 76.66 | 0.76 | 0.00 |
| | G | 48.49 | 121.80 | 10.00 | 111.80 | 37.39 | 0.62 | −1.53 | 77.11 | 0.73 | 0.00 |
| | SW | 6.51 | 7.68 | 5.33 | 2.35 | 0.41 | 0.06 | −0.32 | 6.28 | 1.00 | 0.00 |
| | SA | 41.98 | 54.47 | 28.94 | 25.53 | 4.55 | 0.28 | −0.15 | 10.83 | 0.99 | 0.00 |
| | SLWR | 1.31 | 1.54 | 1.08 | 0.46 | 0.08 | 0.24 | −0.26 | 6.33 | 0.99 | 0.00 |
| Top leaf | TMLL | 92.50 | 120.70 | 60.87 | 59.83 | 9.93 | 0.14 | −0.10 | 10.74 | 1.00 | 0.00 |
| | TMLP | 244.15 | 323.09 | 155.15 | 167.94 | 27.39 | 0.17 | −0.14 | 11.22 | 1.00 | 0.00 |
| | TSLP | 227.34 | 313.21 | 135.03 | 178.18 | 29.84 | 0.21 | −0.10 | 13.13 | 1.00 | 0.00 |
| | TSLL | 82.61 | 110.55 | 52.49 | 58.06 | 9.97 | 0.18 | −0.05 | 12.07 | 1.00 | 0.00 |
| | TMLA | 3222.58 | 5492.32 | 1199.45 | 4292.87 | 778.11 | 0.38 | −0.21 | 24.15 | 0.99 | 0.00 |
| | TSLW | 50.24 | 75.63 | 25.36 | 50.27 | 8.74 | 0.29 | −0.25 | 17.39 | 0.99 | 0.00 |
| | TMLW | 51.12 | 78.04 | 25.53 | 52.50 | 8.99 | 0.21 | −0.32 | 17.59 | 0.99 | 0.00 |
| | TSLA | 2905.73 | 5179.34 | 393.04 | 4786.30 | 810.24 | 0.45 | −0.17 | 27.88 | 0.98 | 0.00 |
| | TMLLWR | 1.86 | 2.95 | 1.22 | 1.73 | 0.32 | 0.76 | 0.32 | 16.91 | 0.96 | 0.00 |
| | TSLLWR | 1.67 | 2.33 | 1.21 | 1.12 | 0.19 | 0.56 | 0.08 | 11.63 | 0.98 | 0.00 |
| Middle | MMLP | 268.67 | 345.59 | 185.72 | 159.86 | 26.18 | 0.01 | −0.15 | 9.74 | 1.00 | 0.18 |
| leaf | MMLA | 4203.03 | 6730.12 | 1789.38 | 4940.74 | 853.49 | 0.23 | −0.18 | 20.31 | 1.00 | 0.00 |
| | MSLL | 92.36 | 118.13 | 61.52 | 56.61 | 9.53 | −0.05 | −0.07 | 10.31 | 1.00 | 0.01 |
| | MSLP | 266.62 | 353.49 | 171.24 | 182.25 | 29.73 | 0.02 | −0.06 | 11.15 | 1.00 | 0.23 |
| | MMLL | 96.27 | 122.07 | 66.89 | 55.18 | 9.05 | −0.01 | 0.00 | 9.40 | 1.00 | 0.06 |
| | MMLLWR | 1.51 | 2.19 | 1.13 | 1.06 | 0.18 | 0.96 | 1.02 | 12.14 | 0.95 | 0.00 |
| | MSLW | 64.37 | 91.25 | 36.55 | 54.71 | 9.04 | 0.00 | −0.15 | 14.05 | 1.00 | 0.28 |
| | MSLA | 4203.63 | 6950.11 | 1627.54 | 5322.57 | 937.13 | 0.28 | −0.17 | 22.29 | 0.99 | 0.00 |
| | MMLW | 63.98 | 91.32 | 35.27 | 56.05 | 8.95 | −0.10 | −0.06 | 13.99 | 1.00 | 0.02 |
| | MSLLWR | 1.44 | 1.94 | 1.10 | 0.84 | 0.14 | 0.90 | 0.94 | 9.58 | 0.95 | 0.00 |
| Bottom | BSLP | 264.71 | 365.43 | 151.88 | 213.55 | 33.45 | −0.17 | 0.02 | 12.64 | 1.00 | 0.00 |
| leaf | BMLL | 92.65 | 122.66 | 58.60 | 64.06 | 10.06 | −0.21 | 0.16 | 10.86 | 1.00 | 0.00 |
| | BMLP | 267.07 | 356.95 | 161.94 | 195.00 | 30.49 | −0.15 | 0.13 | 11.42 | 1.00 | 0.00 |
| | BSLL | 89.34 | 119.90 | 53.06 | 66.84 | 10.73 | −0.24 | 0.12 | 12.02 | 1.00 | 0.00 |
| | BMLW | 65.59 | 91.01 | 35.12 | 55.90 | 8.79 | −0.28 | 0.21 | 13.41 | 0.99 | 0.00 |
| | BMLA | 4229.73 | 6892.83 | 1538.57 | 5354.26 | 900.79 | 0.09 | −0.13 | 21.30 | 1.00 | 0.02 |
| | BSLW | 65.14 | 91.30 | 33.09 | 58.21 | 9.26 | −0.18 | −0.01 | 14.21 | 1.00 | 0.00 |
| | BSLA | 4202.55 | 7141.52 | 1216.28 | 5925.24 | 987.13 | 0.11 | −0.18 | 23.49 | 1.00 | 0.01 |
| | BSLLWR | 1.37 | 1.75 | 1.12 | 0.63 | 0.10 | 0.70 | 0.56 | 7.36 | 0.97 | 0.00 |
| | BMLLWR | 1.41 | 1.90 | 1.09 | 0.81 | 0.13 | 0.79 | 0.78 | 9.18 | 0.96 | 0.00 |
| Petiole | PLM | 17.40 | 25.76 | 7.90 | 17.86 | 2.88 | 0.13 | −0.05 | 16.53 | 1.00 | 0.00 |
| length | PLB | 16.50 | 24.94 | 7.16 | 17.78 | 2.89 | 0.14 | −0.09 | 17.49 | 1.00 | 0.00 |
| | PLT | 14.91 | 23.52 | 5.40 | 18.12 | 2.97 | 0.14 | −0.10 | 19.90 | 1.00 | 0.00 |
| Petiole | PAT | 53.50 | 89.42 | 21.94 | 67.48 | 12.59 | 0.34 | −0.31 | 23.53 | 0.99 | 0.00 |
| angle | PAM | 49.20 | 78.42 | 21.34 | 57.08 | 10.34 | 0.33 | −0.26 | 21.01 | 0.99 | 0.00 |
| | PAB | 45.28 | 71.22 | 20.18 | 51.04 | 9.24 | 0.27 | −0.19 | 20.41 | 0.99 | 0.00 |
| Quality | PC | 44.92 | 49.96 | 39.75 | 10.21 | 1.88 | −0.10 | −0.32 | 4.19 | 1.00 | 0.00 |
| | OC | 18.77 | 22.44 | 15.18 | 7.26 | 1.33 | −0.01 | −0.31 | 7.06 | 1.00 | 0.01 |
| | POC | 63.64 | 68.37 | 58.92 | 9.45 | 1.72 | 0.05 | −0.29 | 2.71 | 1.00 | 0.01 |
| Flower | FC | 1.29 | 2.00 | 1.00 | 1.00 | 0.45 | 0.93 | −1.10 | 34.72 | 0.58 | 0.00 |

Note:
SWPP: seed weight per plant; PNPP: pod number per plant; SNPP: seed number per plant; 100-SW: 100 seed weight; FTM: flowering to maturation; MT: maturation time; FT: flowering time; IN: internode number; SD: stem diameter; PH: plant height; POH: podding habit; LG: lodging; GH: growth habit; BL: branch length; BIN: branch internode; BD: branch diameter; EBN: effective branch number; BA: branch angle; MPN: moldy pod number; MPNR: moldy pod number ratio; FSPNPP: four seeded pod number per plant; BPN: branch pod number; FSR: four seed ratio; PW: pod width; PL: pod length; FPH: first pod height; SPN: stem pod number; SP: seed perimeter; SL: seed length; B: blue; R: red; G: green; SW: seed width; SA: seed area; SLWR: seed length to width ratio; TMLL: top middle leaf length; TMLP: top middle leaf perimeter; TSLP: top side leaf perimeter; TSLL: top side leaf length; TMLA: top middle leaf area; TSLW: top side leaf width; TMLW: top middle leaf width; TSLA: top side leaf area; TMLLWR: top middle leaf length width ratio; TSLLWR: top side leaf length width ratio; MMLP: middle middle leaf perimeter; MMLA: middle middle leaf area; MSLL: middle side leaf length; MSLP: middle side leaf perimeter; MMLL: middle middle leaf length; MMLLWR: middle middle leaf length width ratio; MSLW: middle side leaf width; MSLA: middle side leaf area; MMLW: middle middle leaf width; MSLLWR: middle side leaf length width ratio; BSLP: bottom side leaf perimeter; BMLL: bottom middle leaf length; BMLP: bottom middle leaf perimeter; BSLL: bottom side leaf length; BMLW: bottom middle leaf; BMLA: bottom middle leaf area; BSLW: bottom side leaf width; BSLA: bottom side leaf area; BSLLWR: bottom side leaf length width ratio; BMLLWR: bottom middle leaf length width ratio; PLM: petiole length middle; PLB: petiole length bottom; PLT: petiole length top; PAT: petiole angle top; PAM: petiole angle middle; PAB: petiole angle bottom; PC: protein content; OC: oil content; POC: protein and oil content; FC: flower color.

TABLE 3
Correlation between the seed weight per plant, the seed number per plant,
the pod number per plant, the 100 seed weight and other agronomic traits

| Agronomic | | | | 100- | Agronomic | | | | 100- |
|---|---|---|---|---|---|---|---|---|---|
| traits | SWPP | SNPP | PNPP | SW | traits | SWPP | SNPP | PNPP | SW |
| 100-SW | 0.19 | −0.31 | −0.25 | | TMLA | 0.10 | 0.02 | −0.03 | 0.16 |
| PNPP | 0.77 | 0.88 | | −0.25 | TMLL | 0.20 | 0.14 | 0.12 | 0.10 |
| SNPP | 0.86 | | 0.88 | −0.31 | TMLLWR | 0.10 | 0.14 | 0.18 | −0.1 |
| SWPP | | 0.86 | 0.77 | 0.19 | TMLP | 0.15 | 0.07 | 0.04 | 0.15 |
| GH | −0.04 | 0.01 | 0.03 | −0.08 | TMLW | 0.03 | −0.04 | −0.09 | 0.16 |
| IN | 0.41 | 0.43 | 0.51 | −0.06 | TSLA | 0.11 | 0.04 | −0.01 | 0.15 |
| LG | 0.29 | 0.33 | 0.37 | −0.10 | TSLL | 0.19 | 0.11 | 0.08 | 0.15 |
| PH | 0.38 | 0.38 | 0.45 | −0.01 | TSLLWR | 0.06 | 0.08 | 0.12 | −0.07 |
| POH | 0.21 | 0.26 | 0.24 | −0.13 | TSLP | 0.15 | 0.07 | 0.03 | 0.16 |
| SD | 0.51 | 0.39 | 0.42 | 0.21 | TSLW | 0.09 | 0.02 | −0.03 | 0.15 |
| B | 0.04 | −0.12 | −0.15 | 0.33 | PAT | 0.01 | 0.09 | 0.06 | −0.15 |
| G | 0.05 | −0.12 | −0.15 | 0.34 | MMLA | 0.21 | 0.13 | 0.06 | 0.15 |
| R | 0.06 | −0.10 | −0.15 | 0.34 | MMLL | 0.26 | 0.21 | 0.16 | 0.08 |
| SA | 0.02 | −0.27 | −0.13 | 0.57 | MMLLWR | 0.03 | 0.08 | 0.12 | −0.13 |
| SL | 0.00 | −0.22 | −0.06 | 0.42 | MMLP | 0.23 | 0.16 | 0.10 | 0.13 |
| SLWR | 0.01 | −0.03 | 0.07 | 0.09 | MMLW | 0.17 | 0.09 | 0.02 | 0.16 |
| SP | 0.02 | −0.31 | −0.17 | 0.63 | MSLA | 0.22 | 0.13 | 0.07 | 0.16 |
| SW | −0.01 | −0.22 | −0.15 | 0.39 | MSLL | 0.28 | 0.20 | 0.15 | 0.14 |
| BPN | 0.73 | 0.83 | 0.93 | −0.22 | MSLLWR | 0.07 | 0.10 | 0.13 | −0.1 |
| FPH | 0.09 | −0.01 | −0.03 | 0.19 | MSLP | 0.23 | 0.15 | 0.09 | 0.15 |
| FSPNPP | 0.15 | 0.12 | 0.02 | 0.07 | MSLW | 0.18 | 0.09 | 0.03 | 0.16 |
| FSR | 0.02 | −0.04 | −0.15 | 0.12 | BMLA | 0.35 | 0.26 | 0.22 | 0.16 |
| MPN | 0.34 | 0.21 | 0.28 | 0.23 | BMLL | 0.37 | 0.30 | 0.28 | 0.11 |
| MPNR | 0.05 | −0.12 | −0.08 | 0.34 | BMLLWR | 0 | 0.04 | 0.08 | −0.11 |
| PL | 0.11 | −0.11 | −0.16 | 0.43 | BMLP | 0.34 | 0.25 | 0.22 | 0.16 |
| PW | −0.11 | −0.39 | −0.28 | 0.56 | BMLW | 0.33 | 0.23 | 0.19 | 0.17 |
| SPN | 0.59 | 0.68 | 0.79 | −0.21 | BSLA | 0.37 | 0.27 | 0.23 | 0.17 |
| PLB | 0.29 | 0.18 | 0.20 | 0.21 | BSLL | 0.4 | 0.3 | 0.28 | 0.16 |
| PLM | 0.19 | 0.08 | 0.07 | 0.20 | BSLLWR | −0.01 | 0.01 | 0.05 | −0.06 |
| PLT | 0.20 | 0.07 | 0.07 | 0.23 | BSLP | 0.37 | 0.28 | 0.25 | 0.17 |
| PAB | 0.02 | 0.08 | 0.09 | −0.12 | BSLW | 0.35 | 0.26 | 0.22 | 0.17 |
| PAM | 0.08 | 0.15 | 0.17 | −0.12 | BA | 0.04 | 0.04 | 0.08 | 0 |
| FT | 0.40 | 0.34 | 0.42 | 0.12 | BD | 0.52 | 0.39 | 0.43 | 0.21 |
| FTM | 0.10 | −0.01 | 0.19 | 0.18 | BIN | 0.55 | 0.58 | 0.68 | −0.11 |
| MT | 0.25 | 0.12 | 0.33 | 0.21 | BL | 0.55 | 0.61 | 0.68 | −0.16 |
| OC | −0.04 | −0.07 | −0.10 | 0.05 | EBN | 0.53 | 0.55 | 0.65 | −0.07 |
| PC | −0.07 | −0.16 | −0.07 | 0.20 | FC | −0.05 | −0.07 | −0.09 | 0.06 |
| POC | −0.11 | −0.23 | −0.15 | 0.26 | | | | | |

TABLE 4
Accuracy of GS prediction of soybean yield in the literature

| | Prediction | |
|---|---|---|
| Model | accuracy | References |
| rrBLUP | 0.26 | Stewart-Brown et al., 2019 |
| GBLUP | 0.63 | Alexandra et al., 2017 |
| rrBLUP + Haploid block | 0.48 | Yoosefzadeh-Najafabadi et |
| | | al., 2022 |
| Nonparametric random | 0.60 | Vuk Ðor [symbol] ević et al., 2019 |
| forest | | |
| Bayes B | 0.72 | Matei et al., 2018 |
| RKHS + BayesB | 0.61 | Xavier et al., 2016 |
| rrBLUP | 0.64 | Bandillo et al., 2022 |
| rrBLUP; BayesB; Bayes | 0.69 | Beche et al., 2021 |
| rrBLUP | 0.70 | Jean et al., 2021 |
| SoyDNGP | 0.70 | Gao et al., 2023 |

Note:
PKHS: reproducing kernel space

The above description is only a specific implementation mode of the disclosure, but the protection scope of the disclosure is not limited thereto. Any modifications, equivalent substitutions and improvements made by those skilled in the art within the technical scope disclosed by the disclosure and within the spirit and principle of the disclosure should be covered by the protection scope of the disclosure.

What is claimed is:

1. A method for constructing a genomic selection model based on a multi-trait phenotyping model, comprising:
based on a multi-trait model established by phenotypic data, using predicted values or estimated breeding values obtained by a genomic selection model as input data of a machine learning phenotypic prediction model to predict final phenotypic values for line selection; and
establishing a multi-trait machine learning phenotyping model to obtain linear or nonlinear relationships between plant phenotypic traits, and based on the linear or nonlinear relationships between the plant phenotypic traits, combining the genomic selection model to obtain a predicted value and an estimated breeding value of each trait.

2. The method according to claim 1, comprising:
step 1: establishing the multi-trait machine learning phenotyping model; and
step 2: combining the multi-trait machine learning phenotyping model with the genomic selection model.

3. The method according to claim 2, wherein the step 1 comprises:
making an order of the phenotypic data of respective trait input into the multi-trait machine learning phenotyping model correspond to an order of predicted values and estimated breeding values of the respective traits obtained by the genomic selection model, to ensure data consistency and validity of the linear or nonlinear relationships between the plant phenotypic traits.

4. The method according to claim 2, wherein the establishing the multi-trait machine learning phenotyping model specifically comprises the following steps:
(1) identifying and cleaning missing and abnormal values in the phenotypic data;
(2) randomly rearranging an order of lines in the phenotypic data;
(3) separating a target trait in the phenotypic data and normalizing remaining traits as feature values of the target trait to obtain a normalized trait feature value of each line;
(4) using the normalized trait feature value of each line as input, and training a machine learning phenotyping model according to machine learning model parameters and hyperparameters; and according to performance measurement of predicted phenotypic value results and target trait values, further adjusting various parameters, thereby storing a phenotypic prediction model with best performance measurement as the multi-trait machine learning phenotyping model.

5. The method according to claim 2, wherein the combining the multi-trait machine learning phenotyping model with the genomic selection model specifically comprises the following steps:
1) performing data cleaning, processing and normalization on the predicted values and the estimated breeding values;
2) taking the predicted values or the estimated breeding values after the data cleaning, processing and normalization as feature values of a target trait; and
3) loading a phenotypic prediction model and inputting the feature values for prediction, thereby enabling the phenotypic prediction model to perform prediction based on the input feature values and output final phenotypic prediction values close to the target trait.

6. A system for constructing a genomic selection model based on a multi-trait phenotyping model according to the method of claim 1, comprising:
a data preprocessing unit, configured to receive and process the phenotypic data, including identifying and cleaning missing and abnormal values in the phenotypic data, and randomly rearranging an order of lines;
a data normalization unit, configured to normalize non-target traits in the phenotypic data to obtain normalized trait feature values as feature values and separate a target trait;
a machine learning training unit, configured to train a machine learning phenotyping model according to the normalized trait feature values by setting model parameters and hyperparameters;
a genomic selection model unit, configured to use genomic markers to perform genetic evaluation of individuals to obtain genomic estimated breeding values; and
a model integration unit, configured to combine the machine learning phenotyping model and the genomic selection model to perform data cleaning, processing, and normalization of predicted values and estimated breeding values.

7. The system according to claim 6, wherein the machine learning training unit is configured to adjust the machine learning phenotyping model according to performance measurement of predicted phenotypic value results and target trait values, and store a phenotypic prediction model with best performance measurement.

8. The system according to claim 7, wherein the model integration unit is configured to use the machine learning phenotypic prediction model, input normalized feature values for prediction, and output final phenotypic prediction values close to the target trait.
